(19) United States
(12) Patent Application Publication
(10) Pub. No.: US 2007/0179163 A1
(43) Pub. Date: Aug. 2, 2007

(54) Process for the preparation of risperidone

(76) Inventors: Guntu Srinivasa Rao, Karnataka (IN); Basavapatna N. Prasanna Kumar, Karnataka (IN); Sulur G. Manjunatha, Karnataka (IN)

Correspondence Address: WELSH & KATZ, LTD, 120 S RIVERSIDE PLAZA, 22ND FLOOR, CHICAGO, IL, 60606, US

(21) Appl. No.: 10/572,829

(22) PCT Filed: Sep. 24, 2004
(86) PCT No.: PCT/IN04/00303

§ 371 Date: Nov. 13, 2006

(30) Foreign Application Priority Data
Sep. 26, 2003 (IN) 1209/DEL/2003

(51) Int. Cl. A61K 31/519; C07D 487/04

(52) U.S. Cl. 514/259.41; 544/281

(57) ABSTRACT

A process is provided for the preparation of risperidone of Formula (1); which process comprises reacting, in a condensation reaction, 6-fluoro-3-(4-piperidinyl)-1,2-benzisoxazole monohydrochloride of Formula (2) with 3-(2-chloroethyl)-6,7,8,9-tetrahydro-2-methyl-4H-pyrido[1,2,a]pyrimidin-4-one monohydrochloride of Formula (3).

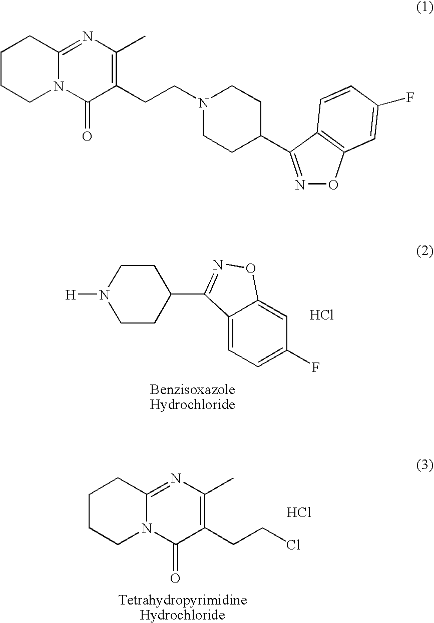

### BACKGROUND OF THE INVENTION

Risperidone is a new serotonin/dopamine antagonist belonging to a new class, the benzisoxazole. The structure of risperidone is shown in Formula-1. It is used for the treatment of schizophrenia and psychotic disorder.

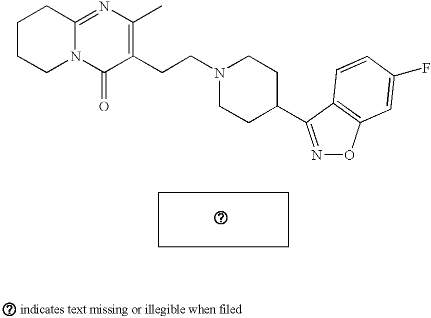

### DESCRIPTION OF THE PRIOR ART

Risperidone was first disclosed in U.S. Pat. No. 4,804,663, according to which it may be prepared by the condensation of the benzisoxazole compound of Formula-2 6-fluoro-3-(4-piperidinyl)-1,2-benzisoxazole in its free base form and the tetahydropyrimidine compound 3-(2-chloroethyl)-6,7,8,9-tetrahydro-2-methyl-4H-pyrido-[1,2-a]pyrimidinone of Formula-3 in its hydrochloride salt form, in the presence of sodium carbonate as a base (condensing agent) and potassium iodide as a catalyst in dimethylformamide (DMF) medium (Scheme-1), followed by standard workup to get crude Risperidone, which is recrystallized in a mixture of dimethylformamaide and isopropyl alcohol to get pure Risperidone with an overall yield of 46%.

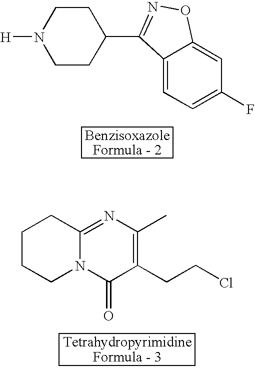

WO-A-02/14256 and WO-A02/12200 disclose another process for producing risperidone, in which the condensation of the intermediates of Formula-2 and Formula-3, in their free base forms, is carried out in isopropyl alcohol or methylethylketone solvent medium, using sodium carbonate as a base (condensing agent). The overall yield as described here is 60%.

Recently, WO-A-01/185731 describes a process for producing risperidone starting from the same two intermediates of Formula-2 and Formula-3, as free base, in the presence of sodium carbonate (condensing agent), but in water medium. Risperidone precipitates as a solid and is filtered and crystallised from dimethylformamide. The overall yield as described here is 65%.

The benzisoxazole of Formula-2, 6-fluoro-3-(4-piperidinyl)-1,2-benzisoxazole and tetrahydropyrimidine of Formula-3, 3-(2-chloroethyl)-6,7,8,9-tetrahydro-2-methyl-4H-pyrido-[1,2-a]pyrimidin-4-one are basic nitrogen heterocyclic derivatives that are solids with low melting points. These two intermediates, in particular the tetrahydropyrimidine of Formula-3, are not stable, on account of their susceptibility to aerial oxidation. Therefore, these intermediates are usually isolated as acid addition salts, and are purified and stored as their acid addition salts, for example their hydrochloride salts. According to the above prior art processes, these acid addition salts have to be converted to the free base forms from the hydrochloride salts, before being subjected to condensation. These steps involve additional operations, which consume time and energy. Also, it is observed that impurities are formed while performing the set-free of said hydrochloride.

The present invention addresses these drawbacks and provides a simple and efficient process for producing risperidone from the stable hydrochloride salts of the two intermediates of Formula-2 and Formula-3. Advantageously, the present invention allows risperidone to be produced by an easily operated process with minimal operation steps and a reduced effluent load.

### SUMMARY OF THE INVENTION

Accordingly, the present invention provides a process for the preparation of risperidone of Formula-1: 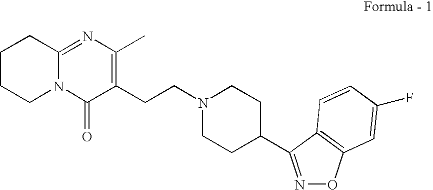
which process comprises reacting, in a condensation reaction, 6-fluoro-3-(4-piperidinyl)-1,2-benzisoxazole monohydrochloride of Formula-2 with 3-(2-chloroethyl)-6,7,8,9-tetrahydro-2-methyl-4H-pyrido[1,2,a]pyrimidin-4-one monohydrochloride of Formula-3:

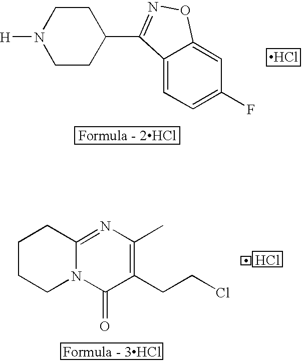

In a first embodiment, the condensation reaction is carded out in the presence of a base (condensing agent), in a solvent medium of water, one or more water-miscible solvents or a mixture of water and one or more water-miscible solvents, and the process comprises:

a) carrying out the condensation reaction at a temperature in the range from 25 to 90° C.;

(b) after completion of the condensation reaction, diluting the condensation reaction mass with ice-cold water to precipitate risperidone;

(c) filtering and drying the precipitated risperidone to obtain crude risperidone; and

(d) crystallizing the crude risperidone in an aqueous solvent to produce pure risperidone.

In a second embodiment, the condensation reaction is carded out in the presence of a base (condensing agent), in a solvent medium of water, one or more water-miscible solvents or a mixture of water and one or more water-miscible solvents, and the process comprises:

a) carrying out the condensation reaction at a temperature in the range from 25 to 90° C.;

(b) after completion of the condensation reaction, then reaction mass is cooled to room temperature and diluting the condensation reaction mass with water to precipitate risperidone;

(c) extracting the precipitated risperidone of step (b) with a water-immiscible solvent;

(d) optionally subjecting the water-immiscible solvent extract to acid-base work-up followed by extraction with a water-immiscible solvent;

(e) concentrating the extract resulting from step (c) or optional step (d) under reduced pressure to produce crude risperidone; and

(f) crystallizing the crude risperidone in an aqueous solvent to produce pure risperidone.

### DETAILED DESCRIPTION OF THE INVENTION

According to the first part of the process of the invention, the intermediates of Formula-2 and Formula-3, as their hydrochloride salts, are used for the condensation reaction, to form risperidone according to the Scheme 1:

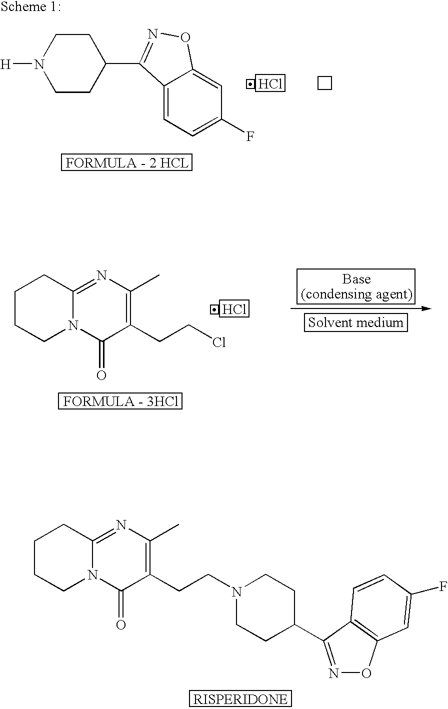

The condensation reaction is carried out in a solvent medium. The solvent medium may be water or one or more water-miscible organic solvents, or a mixture of water and one or more water-miscible organic solvents. Preferably the solvent medium is water or a mixture of water and acetonitrile. Most preferably, the solvent medium is a mixture of water and acetonitrile.

The base (condensing agent) used according to the present invention may be an inorganic salt such as the carbonate, bicarbonate or hydroxide of an alkali metal or alkaline earth metal. Preferred as base is sodium carbonate or potassium carbonate, and most preferred as base is sodium carbonate.

The mole ratio of the base (condensing agent) with respect to the hydrochloride salt of the compound of Formula-2 may be from 2.0:1 to 5.0:1, and more preferably is from 4.0:1 to 4.6:1. Most preferably, the rate is 4.3:1.

The condensation reaction is carried out according to the present invention by dissolving or suspending both of the reactants and reagent in the solvent medium. The sequence of addition of the reactants and reagent is very important. The most preferred sequence is to dissolve or suspend the base (condensing agent) in a solvent medium as described above (preferably water or acetonitrile, more preferably acetonitrile), and then to add to this the hydrochloride salt of the compound of Formula-2. The hydrochloride salt of the compound of Formula -3 is dissolved in a solvent medium as described above (preferably water) and added to the reaction mixture.

Preferably, the solution of the hydrochloride salt of the compound of Formula-3 is added over a period of 1 to 5 hours, and the most preferably is added over a period of 4 to 5 hours. The slow addition of the solution of the hydrochloride of the compound of Formula-3 to the reaction mixture is to avoid the decomposition of the intermediate of Formula-3 under the reaction conditions, and thus enhances the yield and quality of the product risperidone.

The temperature of the reaction mixture during the addition of the solution of the hydrochloride salt of the compound of Formula- 3 is maintained in the range from 25 to 90° C. The temperature of the solution of the hydrochloride salt of the compound of Formula-3 being added is also preferably maintained in this temperature range.

Thus, the condensation reaction is carried out at a temperature in the range from 25 to 90° C., preferably in the range from 40 to 90° C., and more preferably in the range from 50 to 75° C.

After the completion of the addition of the solution of the hydrochloride salt of the compound of Formula-3 the reaction mixture is maintained in the range from 25 to 90° C., preferably in the range from 40 to 90< C., and more preferably in the range from 50 to 75° C., for an additional 2 to 10 hours, and preferably for an additional 4 to 8 hours. Most preferably the reaction mixture is stirred at the same temperature as that of the reaction mixture during the addition of the solution of the hydrochloride salt of the compound of Formula-3, for the additional hours.

Finally the product is isolated by standard work-up, preferably by work-up (i) or (ii) as explained further below, and crystallised to produce pure risperidone as a crystalline solid:

(i) A typical work-up may comprise of diluting the reaction mixture with ice-cold water to precipitate risperidone, filtering and drying the precipitated residue to obtain crude risperidone.

(ii) Alternatively, the reaction mixture is cooled to room temperature and diluted with water to precipitate risperidone, and the precipitated risperidone is then extracted with a water-immiscible organic solvent such as methylene dichloride (i.e. dichloromethane), ethylene chloride, dichloroethane, ethyl acetate, toluene, benzene or chloroform, preferably methylene dichloride, to produce an organic extract. The organic extract is then worked up according to Method A or Method B explained below.

According to Method A, the organic extract (preferably methylene dichloride) is washed with water, treated with activated carbon, and finally concentrated under reduced pressure to obtain crude risperidone.

According to Method B, the organic extract (preferably methylene dichloride) is purified by typical acid-base work-up, preferably as follows: The organic extract (preferably methylene dichloride) is extracted with aqueous acid such as 10-25% aqueous acid, preferably 10-15% aqueous acid, for example formic acid, acetic acid, hydrochloric acid, hydrobromic acid or tartaric acid. Preferred is 10-15% aqueous hydrochloric acid. The aqueous acidic extract is optionally, but preferably, washed with organic solvent such as toluene, methylene dichloride, dichloroethane or ethyl acetate, or mixtures thereof, preferably methylene dichloride. The aqueous acidic extract is cooled to 15-25° C. and the pH adjusted to 8-9 at 15-25° C. by addition of a base such as aqueous sodium or potassium hydroxide, aqueous sodium or potassium carbonate or bicarbonate, or liquor ammonia solution. Most preferred as base is liquor ammonia solution. The resulting reaction mixture is extracted with a water-immiscible organic solvent such as methylene dichloride, ethylene chloride or chloroform, preferably with methylene dichloride. The organic (preferably methylene dichloride) extract is washed with water, treated with activated carbon and finally concentrated under reduced pressure to obtain crude risperidone worked up according to Method B.

Then, the crude risperidone obtained from work-up (i) or from Method A or B in work-up (ii) is crystallised in an aqueous solvent, preferably 5-20% aqueous solvent, selected from aqueous acetone, aqueous methyl ethyl ketone, aqueous methyl isobutyl ketone, aqueous acetonitrile and aqueous dimethylformamide, preferably aqueous acetone, especially 10% aqueous acetone, to produce pure risperidone as a crystalline solid. By this method, it is possible to obtain directly a pharmaceutically acceptable grade of risperidone, for example having purity greater than 99% (as deterrmined by HPLC).

The crystallisation is carried out in known manner, for example by dissolving the crude risperidone in the aqueous solvent at 50-70° to produce a clear solution, treating the solution wit activated carbon, filtering, cooling to 0-5° C., and then separating the pure risperidone by filtration.

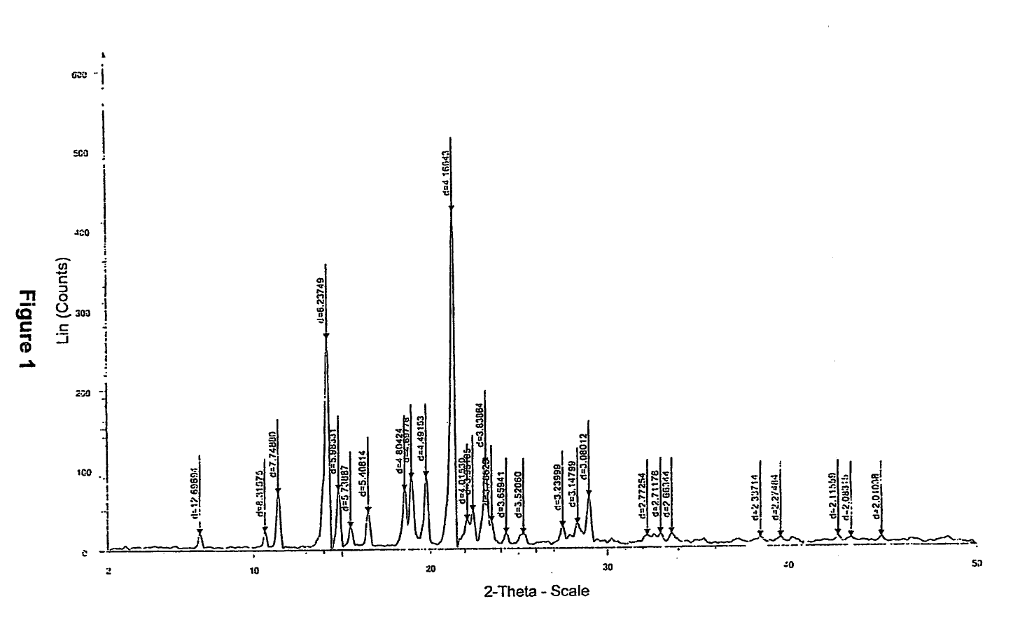

When crystallised from an aqueous ketonic solvent selected from aqueous acetone, aqueous methyl ethyl ketone and aqueous methyl isobutyl ketone, crystalline risperidone is obtained having a polymorphic form identical to that of risperidone obtained from /the inventors' recrystallizing process as disclosed in U.S. Pat. No. 4,804,663 i.e. crystallization from IPA/DMF mixture. This is confirmed by the X-ray diffraction (XRD) analysis as shown in FIG. 1. This polymorphic form is designated as Form B in US-A-2002/0115672 (Mayers) and as Form A in WO-A-02/12200 (Teva). As shown by FIG. 1, this polymorphic form has peaks at about 6.956, 10.630, 11.410, 14.188, 14.794, 15.428, 16.377, 18.453, 18.875, 19.750, 21.309, 22.121, 22.427, 23.152, 23.477, 24.303, 25.77, 27.507, 28.328, 28.965, 32.262, 33.005, 33.622, 38.488, 39.585, 42.705, 43.404 and 45.059±0.2 degrees two theta.

Risperidone base, thus crystallized, may be converted to pharmaceutically acceptable non-toxic acid addition salts such as hydrochloride, tartrate or palmate salts, by conventional methods.

The benzisoxazole compound of Formula-2 is preferably prepared according to the procedure described in the U.S. Pat. No. 4,355,037.

The tetrahydropyrimidine compound of Formula-3 is preferably prepared by hydrogenation of the corresponding pyrimidine derivative 3-(2-chloroethyl)-2-methyl-4H-pyrido[1,2,a]pyrimidin-4-one, preferably in methanol using a Raney nickel catalyst according to Scheme-2.

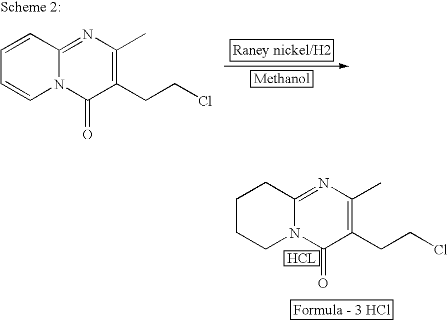

The preferred hydrogenation reaction temperature is 28-35° C., and preferred hydrogen pressure 70-80 psi. The pyrimidine derivative itself prepared according to known procedures by the condensation of 2-aminopyridine with 2-acetylbutyrolactone.

The present invention is further illustrated by the following non-limiting experimental examples:

### EXAMPLES

Experimental Details for Preparation of Risperidone

### Example 1

### Condensation Reaction in Water Medium

6-Fluoro-3-(4-piperidinyl)-1,2-benzisoxazole hydrochloride (Formula-2.HCl, 100 g) is added to a solution of sodium carbonate(180 g) in 400 ml water at 25-30° C. Slowly the reaction mass is warmed to 50-55° C. and then a solution of 3-(2-chloroethyl)-6,7,8,9-tetrahydro-2-methyl-4H-pyrido[1,2-a]pyrimidin-4-one hydrochloride (Formula-3.HCl, 150 g) in water (300 ml) is added gradually over a period of 5 hours at 50-55° C. The reaction mass temperature is maintained further for another 4 hours. The reaction mass is cooled to room temperature and diluted with (200 ml) water the precipitated risperidone is separated by filtration, washed with water (50 ml) and dried to get crude risperidone.

Crude risperidone weight=135 gm

Purity=90-95% (HPLC)

### Example 2

### Condensation Reaction in Water Medium

6-Fluoro-3-(4-piperidinyl)-1,2-benzisoxazole hydrochloride (Formula-2.HCl, 100 g) is added to a solution of sodium carbonate(180 g) in 400 mi water at 25-30° C. Slowly the reaction mass is warmed to 50-55° C. and then a solution of 3-(2-chloroethyl)-6,7,8,9-tetrahydro-2-methyl-4H-pyrido[1,2-a]pyrimidin-4-one hydrochloride (Formula-3.HCl, 150 g) In water (300 ml) is added gradually over a period of 5 hours at 50-55° C,. The reaction mass temperature Is maintained further for another 4 hours. The reaction mass is cooled to room temperature and diluted with (200 ml) water the precipitated risperidone is extracted with dichloromethane (3×450 ml). The dichloromethane extract is used for further work-up according to Method A or Method B, as given below to get crude risperidone.

### Example 3

### Condensation Reaction in Mixture of Water and Water-Miscible Solvents

6-fluoro-3-(4-piperidinyl)-1,2 benzisoxazole hydrochloride (100 g) is added to a suspension of sodium carbonate (180 g) in acetonitle (500 ml) at 25-30° C. Slowly, the reaction mass is warmed to 70-75° C. and then a solution of 3-(2-chloroethyl)-6,7,8,9-tetrahydro-2-methyl-4H-pyrido[1,2-a]pyrimidin-4-one hydrochloride (110 g) in water (200 ml) is added gradually over a period of 4 hours at 70-75° C. The reaction mass is maintained at the same temperature for an additional 4 hours. The reaction mass is then cooled to room temperature and diluted with water (500 ml). The resulting mixture is extracted with dichloromethane (3×450 ml). The dichloromethane extract is worked up as explained for Method A in Example 1 to produce crude risperidone

Method A: The dichloromethane extract is washed with 2×150 ml of water, treated with activated carbon, and concentrated under reduced pressure to produce crude risperidone.

Crude risperidone: 190-200 g

Purity: ˜85-90% (HPLC)

Method B: The dichloromethane extract is extracted with aqueous dilute hydrochloric acid (10%). The aqueous extract is washed with dichloromethane (200 ml) and basified with aqueous ammonia to pH 8.5-9.0. The aqueous mass is extracted with dichloromethane (3×450 ml), and the dichloromethane extract is washed with water, treated with activated carbon and then concentrated under reduced pressure to produce crude risperidone.

Crude risperidone: 180-190 g

Purity: ˜87-92%(HPLC)

### Example 4

### Purification of Crude Risperidone

A) From 10% Aqueous Acetone:

Risperidone crude (100 g) is dissolved in 10% aqueous acetone (700 ml) at 50-55° C., then treated with 10% activated carbon and filtered. The clear filtrate is gradually cooled to 0-5° C. over a period of 45 hours . The crystallized risperidone is separated by filtration and washed with chilled 10% aqueous acetone followed by drying at 50-55° C. under vacuum to get pure risperidone.

Pure risperidone: 75-80 g.

Purity: >99%( HPLC)

B) From 10% aqueous acetonitrile:

Risperidone crude (100 g) is dissolved in 10% aqueous acetonitile (500 ml) at 65-70° C., then treated with 10% activated carbon and filtered. The clear filtrate is gradually cooled to 0-5° C. over a period of 45 hours. The crystallized risperidone is separated by filtration and washed with chilled 10% aqueous acetontrile followed by drying at 50-55° C. under vacuum to get pure risperidone.

Pure risperidone: 80-85 g Purity: >99% (HPLC)

C) From 10% aaueous methvl ethyl ketone:

Risperidone crude (100 g) is dissolved in 10% aqueous methyl ethyl ketone (600 ml) at 65-70° C., then treated with 10% activated carbon and filtered. The clear filtrate is gradually cooled to 0-5° C. over a period of 4-5 hours. The crystallized risperidone is separated by filtration and washed with chilled 10% aqueous methyl ethyl ketone followed by drying at 50-55° C. under vacuum to get pure risperidone.

Pure risperidone: 65-70 g

Purity. >99% (HPLC)

D) From 5% aqueous isobutyl methyl ketone:

Risperidone crude (100 g) is dissolved in 5% aqueous isobutyl methyl ketone (650 ml) at 65-70° C., then treated with 10% activated carbon and filtered. The clear filtrate is gradually cooled to 0-5° C. over a period of 4-5 hours. The crystallized risperidone is separated by filtration and washed with chilled 10% aqueous isobutyl methyl ketone followed by drying at 50-55° C. under vacuum to get pure risperidone.

Pure risperidone: 60-65 g

Purity: >99%( HPLC)

Crude risperidone is prepared using the same procedure as described in Example-3, but using different solvent media and temperature as given in Table-1, Instead of acetonitrile (500 ml) /water (200 ml) at 70-75° C. in Example-3, in the condensation reaction to get crude risperidone. The above isolated crude risperidone is purified as disclosed in Example-4 ;A,B,C and D.

The weights, yields & purities of pure risperidone (samples 1-8) are given in Table 1:

| | | Condensation | | | |
|---|---|---|---|---|---|
| | Solvent used for | reaction | | | |
| SI. | condensation | temperature | Weight | Purity | Yield |
| No | reaction | (° C.) | (g) | (%) | (%) |
| 1 | Water | 50-55 | 121 | 99.34 | 75.8% |
| 2 | Water:DMF | 55-60 | 110 | 99.67 | 68.76% |
| | (1.0:4.6 v/v) | | | | |
| 3 | Water:DMF | 65-70 | 120 | 99.87 | 75% |
| | (1.0:7.0 v/v) | | | | |
| 4 | Water:IPA | 60-65 | 80 | 99.67 | 50% |
| | (1.0:14.0 v/v) | | | | |
| 5 | Water:MeOH | 60-65 | 80 | 99.74 | 50% |
| | (1.0:30.0 v/v) | | | | |
| 6 | Water:ACN | 65-70 | 135 | 99.63 | 81.2% |
| | (1.0:4.0 v/v) | | | | |
| 7 | Ethanol | 65-70 | 115 | 99.72 | 67.5% |
| 8 | DMF | 65-70 | 60 | 98.77 | 37.5% |

### Example 5

### Preparation of 3-(2-chloroethyl)-6,7,8,9-tetrahydro-2-methyl-4H-pyrido[1,2-a]pyrimidin-4-one hydrochloride (Formula-3

### (1):Preparation of 3-(2-chloroethyl)-2-methyl-4H-pyrido[1,2-a]pyrimidin-4-one

2-Aminopyridine (100 g) is added to a solution of toluene (100 ml) and phosphorus oxychloride (365 g) at 0-5° C. and then the temperature is raised to 50-55° C. 2-Acetylbutyrolactone (82 g) is added to the mixture at the same temperature. The temperature is raised to 90-95° C. and maintained for an additional 5 hours. Additional 2-acetylbutyrolactone (82 g) is added at this temperature and the temperature is further maintained for an additional 9-10 hours: Toluene and the excess phosphorus oxychloride is then distilled off under reduced pressure and the residue is quenched over ice-water mixture. The pH of the resulting aqueous mixture is adjusted to 89 with liquor ammonia and the precipitated solid is extracted with dichloromethane (3×200 ml). The organic extract is washed with water and then concentrated under reduced pressure to obtain a residue. The residue is triturated with isopropyl alcohol to produce 3-(2-chloroethyl)-2-methyl-4H-pyrido[1,2-a]pyrimidin-4-one.

### (2):Preparation of 3-(2-chloroethyl)-6,7,8,9-tetrahydro-2-methyl-4H-pyrido[1,2-a]pyrimidin-4-one hydrochloride (Formula-3)

3-(2-Chloroethyl)-2-methyl-4H-pyrido[1,2-a]pyrimidin-4-one(100 gm) is taken in methanol(500 ml) in a pressure reactor and Raney nickel(10 g) added to it. The reactor is pressurised with hydrogen at 70-80 psi and the mixture is stirred at 28-35° C. until the hydrogen absorption ceases (approximately after 6 hours). The Raney nickel catalyst is then filtered. The pH of the filtrate is adjusted to 1.5-2.0 with concentrated hydrochloric acid (50-60 ml). Methanol is then distilled off under reduced pressure and isopropyl alcohol (500 ml) is added to the residue. The resulting slurry is cooled to 0-5° C. and the precipitated solid is filtered. The solid is washed with cold isopropyl alcohol and dried to produce 3-(2-chloroethyl)-6,7,8,9-tetrahydro-2-methyl-4H-pyrido]1,2-a]pyrimidin-4-one hydrochloride of Formula-3.

Yield: 90 g

Purity: >98% (by HPLC)

1. A process for the preparation of risperidone of Formula 1: 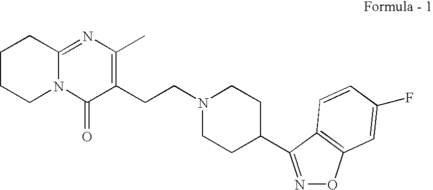
which process comprises reacting, in a condensation reaction, 6-fluoro-3-(4-piperidinyl)-1,2-benzisoxazole monohydrochloride of Formula-2 with 3-(2-chloroethyl)-6,7,8,9-tetrahydro-2-methyl-4H-pyrido[1,2,a]pyrimidin-4-one monohydrochloride of Formula-3:

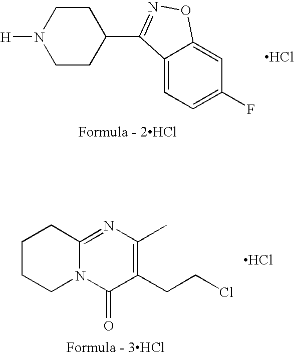

2. A process according to claim 1, wherein the condensation reaction is carried out in the presence of a base (condensing agent), in a solvent medium-of water, one or more water-miscible solvents or a mixture of water and one or more water-miscible solvents, and the process comprises:
a) carrying out the condensation reaction at a temperature in the range from 25 to 90° C.;
(b) after completion of the condensation reaction, diluting the condensation reaction mass with ice-cold water to precipitate risperidone;
(c) filtering and drying the precipitated risperidone to obtain crude risperidone; and
(d) crystallizing the crude risperidone in an aqueous solvent to produce pure risperidone.

3. A process according to claim 1, wherein the condensation reaction is carried out in the presence of a base (condensing agent), in a solvent medium of water, one or more water-miscible solvents or a mixture of water and one or more water-miscible solvents, and the process comprises:
a) carrying out the condensation reaction at a temperature in the range from 25 to 90° C.;
(b) after completion of the condensation reaction, then reaction mass is cooled to room temperature and diluting the condensation reaction mass with water to precipitate risperidone;
(c) extracting the precipitated risperidone of step (b) with a water-immiscible solvent;
(d) optionally subjecting the water-immiscible solvent extract to acid-base work-up followed by extraction with a water-immiscible solvent;
(e) concentrating the extract resulting from step (c) or optional step (d) under reduced pressure to produce crude risperidone; and
(f) crystallizing the crude risperidone in an aqueous solvent to produce pure risperidone.

4. A process according to claims 1, wherein the condensation reaction is carried out in a mixture of water and one or more water-miscible solvents.

5. A process according to claims 1, wherein the condensation reaction is carried out in water as the only solvent.

6. A process according to claims 2, wherein the water-miscible solvent is selected from methanol, ethanol, propanol, isopropanol, acetone, acetonitrile, dimethyl formamide, dimethyl sulfoxide, and mixtures thereof.

7. A process according to claim 1, wherein the condensation reaction is carried out at a temperature in the range from 40 to 90°.

8. A process according to claim 2, wherein the base (condensing agent) is selected from sodium or potassium carbonate, sodium or potassium bicarbonate, and sodium or potassium hydroxide.

9. A process according to claim 8, wherein the base (condensing agent) is sodium carbonate.

10. A process according to claim 3, wherein the water-immiscible solvent is selected from dichloromethane, dichloroethane, chloroform, ethyl acetate, toluene, benzene, and mixtures thereof,

11. A process according to claim 10, wherein the water-immiscible solvent is dichloromethane.

12. A process according to claim 3, wherein the water-immiscible solvent extract is back extracted with 10-15% aqueous acid.

13. A process according to claim 12, wherein the acid is selected group from hydrochloric acid, hydrobromic acid, tartaric acid and acetic acid.

14. A process according to claim 14, wherein the pH of the aqueous acidic extract is adjusted to basic with ammonia and is further extracted into dichloromethane.

15. A process according to claim 14, wherein the pH of the aqueous acidic extract is adjusted to basic with ammonia and is further extracted into dichloromethane.

16. A process according to claim 2, wherein the crude risperidone is crystallized in an aqueous solvent selected from aqueous acetone, aqueous methyl ethyl ketone, aqueous methyl isobutyl ketone, aqueous acetonitrile and aqueous dimethylformamide, to produce pure risperidone.

17. A process according to claim 16, wherein the aqueous solvent is aqueous acetone.

18. A process according to claim 1, wherein the 3-(2-chloroethyl)-6,7,8,9-tetrahydro-2-methyl-4H-pyrido[1,2,a]pyrimidin-4-one monohydrochloride of Formula 3 is prepared starting from 3-(2-chloroethyl)-2-methyl-4H-pyrido[1,2,a]pyrimidin-4-one.

19. A process according to claim 18, wherein the 3-(2-chloroethyl)-2-methyl-4H-pyrido[1,2,a]pyrimidin-4-one is hydrogenated in the presence of a metal catalyst and hydrogen pressure.

20. A process according to claim 19, wherein the metal catalyst is Raney nickel.

21. A process according to claim 20, wherein the hydrogen pressure is 70-80 psi.

22. A process according to claim 21, wherein the hydrogenation reaction temperature is 28-35.

23. (canceled)

24. A process for preparing crystalline risperidone comprising crystallizing the condensed product obtained by reacting 6-fluoro-3-(4-piperidinyl)-1,2-benzisoxazole monohydrochloride with 3-(2-chloroethyl)-6,7,8,9-tetrahydro-2-methyl-4H pyrido[1,2,a]pyrimidin-4-one monohydrochloride or crude risperidone in an aqueous organic solvent.

25. The process according to claim 24, wherein the aqueous organic solvent is selected from aqueous acetone, aqueous methyl ethyl ketone, aqueous methyl isobutyl ketone or mixtures thereof.
